(19) United States
(12) Patent Application Publication
(10) Pub. No.: US 2023/0000446 A1
(43) Pub. Date: Jan. 5, 2023

(54) APPARATUS AND METHOD FOR ESTIMATING LIPID CONCENTRATION

(71) Applicants: SAMSUNG ELECTRONICS CO., LTD., Suwon-si (KR); Korea University Research and Business Foundation, Seoul (KR)

(72) Inventors: Yun S PARK, Suwon-si (KR); Seoung Bum KIM, Seoul (KR); Yong Joo KWON, Yongin-si (KR); Mingu KWAK, Seoul (KR); Yoon Sang CHO, Seoul (KR); Chunghyup MOK, Seoul (KR); Yeol Ho LEE, Uiwang-si (KR); Joon Hyung LEE, Seongnam-si (KR); Kee Won JEONG, Seoul (KR); Jinsoo BAE, Seoul (KR)

(73) Assignees: SAMSUNG ELECTRONICS CO., LTD., Suwon-si (KR); Korea University Research and Business Foundation, Seoul (KR)

(21) Appl. No.: 17/490,714

(22) Filed: Sep. 30, 2021

(30) Foreign Application Priority Data
Jun. 30, 2021 (KR) 10-2021-0085999

(51) Int. Cl. A61B 5/00; A61B 5/145; A61B 5/1455

(52) U.S. Cl. CPC A61B 5/7267; A61B 5/14546; A61B 5/7275; A61B 5/1455

(57) ABSTRACT

An apparatus for estimating lipid concentration is provided. According to an example embodiment, the apparatus may include a training data collector configured to collect, as training data, a reference lipid concentration measured through blood samples of a plurality of users for a predetermined time period and sensor data obtained through light signals detected from the plurality of users for the predetermined time period and a processor configured to perform preprocessing including a moving average and data augmentation on the obtained sensor data, select a valid variable relevant to a change in lipid concentration based on the preprocessed sensor data and the reference lipid concentration, and generate a lipid concentration prediction model based on the selected valid variable.

### CROSS-REFERENCE TO RELATED APPLICATION(S)

This application is based on and claims priority to Korean Patent Application No. 10-2021-0085999, filed on Jun. 30, 2021, in the Korean Intellectual Property Office, the entire disclosure of which is herein incorporated by reference for all purposes.

### BACKGROUND

### 1. Field

The disclosure relates to lipid concentration estimation.

### 2. Description of Related Art

With the aging population, increased medical costs, and a lack of medical personnel for specialized medical services, research is being actively conducted on information technology (IT)-medical convergence technologies, in which IT technology and medical technology are combined. Particularly, monitoring of a health condition of a human body may not be limited to places such as hospitals, but is expanded by mobile healthcare fields that may monitor a user's health condition anywhere (e.g., at home or office or in transit from one place to another place) and anytime in daily life. Some examples of bio-signals, which indicate the health condition of individuals, may include an electrocardiography (ECG) signal, a photoplethysmogram (PPG) signal, an electromyography (EMG) signal, and the like, and various bio-signal sensors are being developed to measure the bio-signals in daily life.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to an aspect of an example embodiment, there is provided an apparatus for estimating lipid concentration, including: a training data collector configured to collect, as training data, a reference lipid concentration measured through blood samples of a plurality of users for a predetermined time period and sensor data obtained through light signals detected from the plurality of users for the predetermined time period; and a processor configured to perform preprocessing, including a moving average and data augmentation, on the obtained sensor data, configured to select a valid variable relevant to a change in lipid concentration based on the preprocessed sensor data and the reference lipid concentration, and configured to generate a lipid concentration prediction model based on the selected valid variable.

The training data collector may further collect, as the training data, metadata including at least one of gender, age, height, weight, body mass index (BMI), skin temperature, or skin humidity of the plurality of users and the processor may select the valid variable based further on the collected metadata.

The processor may perform preprocessing on a sensor data variable obtained over time for each user of the plurality of users using a cumulative weighted moving average, wherein a lower weight may be assigned to data farther from a central point of a predetermined moving average period unit.

The processor may obtain additional sensor data by augmenting data based on the sensor data using a data augmentation technique including Gaussian blur.

The processor may scale a senor data variable using an L-2 norm.

The processor may classify the collected training data into at least two groups based on the reference lipid concentration and select the valid variable by comparing the training data between the classified at least two groups.

The processor may select the valid variable by applying a nonparametric statistical test including a Wilcoxon rank-sum test to the training data in the classified groups.

The processor may select the valid variable using an auto-encoder based on the training data.

The processor may generate the lipid concentration prediction model based further on a machine learning model including at least one of partial least square (PLS), elastic net, random forest, gradient boosting machine (GBM), or XGBoost.

The training data collector may include a light sensor provided in a pixel array, the pixel array including light sources configured to emit light toward an object and detectors configured to detect a light signal through light scattered or reflected from the object.

The processor may drive a light source of a specific pixel and detectors of all pixels in the light sensor.

The processor may sequentially drive light sources of pixels in a specific row of the pixel array and drive detectors in remaining rows of the pixel array while the light sources of the pixel in the specific row are being sequentially driven.

The processor may sequentially drive light sources of all pixels of the pixel array and drive a detector of the same pixel as that of a driven light source while the light sources of all pixels are being sequentially driven.

The processor may generate a personalized lipid concentration prediction model by performing a calibration based on the generated lipid concentration prediction model, a bio-signal obtained through a light signal detected from a specific user, and metadata of the specific user.

According to an aspect of another example embodiment, there is provided a method of estimating lipid concentration, including: collecting, as training data, a reference lipid concentration measured through blood samples of a plurality of users for a predetermined time period and sensor data obtained through light signals detected from the plurality of users for the predetermined time period; performing preprocessing including a moving average and data augmentation on the obtained sensor data; selecting a valid variable relevant to a change in lipid concentration based on the preprocessed sensor data and the reference lipid concentration; and generating a lipid concentration prediction model based on the selected valid variable.

The collecting may include further collecting, as the training data, metadata including at least one of gender, age, height, weight, body mass index (BMI), skin temperature, or skin humidity of the plurality of users and the selecting of the valid variable may include selecting the valid variable based further on the collected metadata.

The performing the preprocessing may include obtaining additional sensor data by augmenting data based on the sensor data using a data augmentation technique including Gaussian blur.

The selecting of the valid variable may include classifying the collected training data into at least two groups based on the reference lipid concentration and selecting the valid variable by comparing the training data between the classified at least two groups.

The selecting of the valid variable by comparing the training data between the classified at least two groups may include selecting the valid variable by applying a nonparametric statistical test including a Wilcoxon rank-sum test to the training data in the classified at least two groups.

The selecting of the valid variable may include selecting the valid variable using an auto-encoder based on the training data.

Other features and aspects will be apparent from the following detailed description, the drawings, and the claims.

### BRIEF DESCRIPTION OF THE DRAWINGS

The above and other aspects, features, and advantages of certain example embodiments of the disclosure will be more apparent from the following description taken in conjunction with the accompanying drawings.

FIG. 1 is a block diagram illustrating an apparatus for estimating lipid concentration according to an example embodiment.

FIG. 2 is a block diagram illustrating an apparatus for estimating lipid concentration according to an example embodiment.

FIGS. 3A, 3B, and 3C are diagrams for explaining a process in which a light source and a detector are driven in a light sensor formed of a pixel array.

FIG. 4 is a block diagram illustrating an apparatus for estimating lipid concentration according to an example embodiment.

FIG. 5 is a flowchart illustrating a method of estimating lipid concentration according to an example embodiment.

FIG. 6 is a flowchart illustrating a method of estimating lipid concentration according to an example embodiment.

FIG. 7 is a diagram illustrating a wearable device according to an example embodiment.

FIG. 8 is a diagram illustrating a smart device according to an example embodiment.

### DETAILED DESCRIPTION

Details of example embodiments are provided in the following detailed description with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience. The disclosure may be understood more readily by reference to the following detailed description of example embodiments and the accompanying drawings. The disclosure may, however, be embodied in many different forms and should not be construed as being limited to the embodiments set forth herein. Rather, these embodiments are provided so that the disclosure will be thorough and complete and will fully convey the concept of the invention to those skilled in the art, and the disclosure will only be defined by the appended claims. Like reference numerals refer to like elements throughout the specification.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. In the specification, unless explicitly described to the contrary, the word “comprise” and variations such as “comprises” or “comprising,” will be understood to imply the inclusion of stated elements but not the exclusion of any other elements. Terms such as “unit” and “module” denote units that process at least one function or operation, and they may be implemented by using hardware, software, or a combination of hardware and software.

Hereinafter, example embodiments of the apparatus and method for estimating lipid concentration will be described in detail with reference to the drawings.

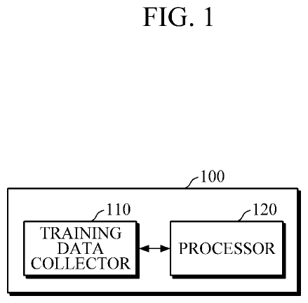

FIG. 1 is a block diagram illustrating an apparatus for estimating lipid concentration according to an example embodiment.

Various example embodiments of the apparatus 100 for estimating lipid concentration may be mounted in various terminals, such as a smartphone, a tablet personal computer (PC), a desktop PC, a notebook PC, a wearable device, and the like. Here, the wearable device may include a watch type, a wristlet type, a wrist band type, a ring type, a glasses type, and a hair band type. However, the disclosure is not limited thereto and the apparatus 100 may be mounted in any hardware manufactured in various forms, e.g., mounted in hardware used in specialized medical institutions.

Referring to FIG. 1, an apparatus 100 for estimating lipid concentration includes a training data collector 110 and a processor 120.

The training data collector 110 may collect reference lipid concentration, metadata of a plurality of users, and sensor data of the plurality of users as training data. In this case, the training data collector 110 may collect, as training data, reference lipid concentration, metadata of a plurality of users, and sensor data of the plurality of users for the same predetermined time period. For example, for a total of 5 days, 7 times a day at a predetermined time interval, a total of 35 reference lipid concentrations, metadata, and sensor data may be collected as training data, but the training data is not limited thereto, and the total predetermined period, number of days of measurement, the predetermined time interval, and the total number of collections may be varied without limitation.

The reference lipid concentration may mean a lipid concentration measured through blood samples of a plurality of users, and in this case, the reference lipid concentration is a result of collecting blood samples from a plurality of users a predetermined number of times for a predetermined time period and may be obtained by measuring the lipid concentration from the collected samples through an external device (not shown).

The metadata may include any one of gender, age, height, weight, BMI, body fat mass, muscle mass, body water content, skin temperature, and skin humidity of a plurality of users. In this case, the metadata may be directly input to the apparatus 100 for estimating lipid concentration by the plurality of users, measured by another configuration included in the apparatus 100, or received from an external device (not shown).

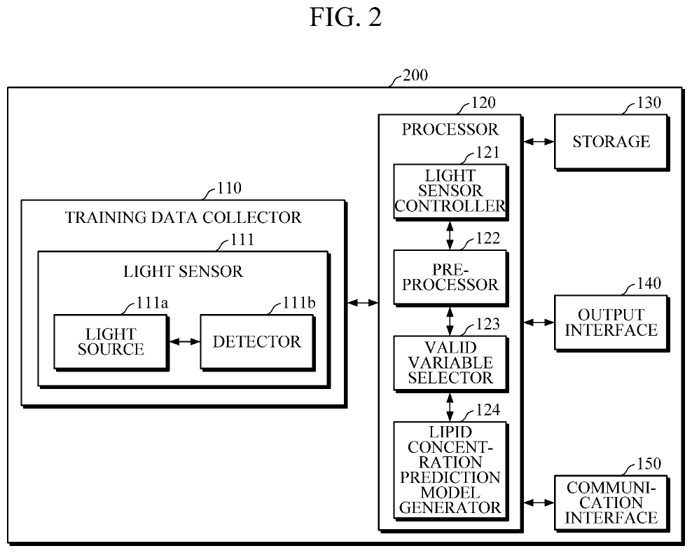

The training data collector 110 may collect, as training data, sensor data obtained through light signals detected from the plurality of users for a predetermined time period. The sensor data may refer to a plurality of light signals detected by each of a plurality of detectors (e.g., 111b in FIG. 2) of a light sensor (e.g., 111 in FIG. 2) that may be included in the training data collector 110.

An electrical, mechanical, wired, and/or wireless connection between the processor 120 and the training data collector 110 may be established. Upon request for generating a lipid concentration model, the processor 120 may control the training data collector 110 and receive sensor data, reference lipid concentration, and metadata from the training data collector 110.

The processor 120 may select a valid variable significant to the change in lipid concentration based on the training data received from the training data collector 110, and generate a lipid concentration prediction model based on the selected valid variable. In this case, lipids may include triglycerides.

Variables may refer to all factors that affect the change in lipid concentration, including metadata variables and sensor data variables.

For example, the metadata variable may mean any one of gender, age, height, weight, BMI, body fat mass, muscle mass, body water content, skin temperature, and skin humidity of a user, but is not limited thereto.

In another example, the sensor data variable may be a feature value of each of the plurality of light signals detected by the light sensor 111, and the feature value may be predetermined. In this case, the feature value may be determined based on an alternating current (AC) component signal and a direct current (DC) component signal of the detected light signal. For example, the feature value may be an average of AC component amplitudes, an average of DC component amplitudes, or an average value obtained by dividing an AC component by a DC component. Alternatively, the feature value may be an area value of the detected light signal, a maximum value, a minimum value, and a statistical value of a maximum value and a minimum value in a differential signal of the light signal, or the like, but is not limited thereto.

The sensor data acquisition process of the training data collector 110 and the preprocessing process, valid variable selection process, and lipid concentration prediction model generation process of the processor 120 will be described in detail with reference to FIG. 2.

FIG. 2 is a block diagram illustrating an apparatus 200 for estimating lipid concentration according to an example embodiment. Referring to FIG. 2, the apparatus 200 for estimating lipid concentration may include a training data collector 110, a processor 120, a storage 130, an output interface 140, and a communication interface 150.

The training data collector 110 may include a light sensor 111. The light sensor 111 may be formed of a pixel array and may obtain sensor data from a plurality of users for a predetermined time period. Each pixel of the pixel array may include a light source 111a configured to emit light to an object and a detector 111b configured to detect a light signal through light scattered or reflected from the object.

Each pixel of the light sensor 111 may detect a plurality of light signals from an object of a user using the light source 111a and the detector 111b. In this case, the light signal may include a photoplethysmography (PPG) signal or an electrocardiography (ECG) signal, but is not limited thereto. As described above in FIG. 1, the sensor data may mean a plurality of light signals detected by a plurality of detectors 111b of the light sensor 111.

In this case, the object may be a region of a wrist surface adjacent to the radial artery, which is an upper area of the wrist where the capillary blood or venous blood passes through, or a body part with a high blood vessel density, e.g., a finger, a toe, an earlobe, etc.

The light source 111a of each pixel may include at least one of a light emitting diode (LED), a laser diode, or a phosphor, but is not limited thereto. In this case, the light source 111a of each pixel may be formed of, for example, an LED array, and each LED may emit light at a different wavelength, such as a green, red, and/or infrared.

The detector 111b of each pixel may include a photodiode, a photo transistor, a photodiode array, a phototransistor array, an image sensor (e.g., a complementary metal oxide semiconductor (CMOS) image sensor), etc.

The light sensor 111 may further include an additional configuration to be used for sensor data acquisition. For example, additional configurations, such as an amplifier configured to amplify an electrical signal output by the detector 111b that has detected by the light signal, or an analog-to-digital converter configured to convert an electrical signal output by the amplifier into a digital signal, may be further included in the light sensor 111. In addition, in a case where the light sensor 111 measures an ECG signal, the light sensor 111 may include a plurality of electrodes.

The processor 120 may include a light sensor controller 121, a preprocessor 122, a valid variable selector 123, and a lipid concentration prediction model generator 124.

The light sensor controller 121 may drive the light sensor 111 in various ways. In this case, for example, the driving method of the light sensor 111, including information on a light source of a pixel to be driven, a duration, light source intensity, a detector of a pixel to be driven, and the like, may be predefined.

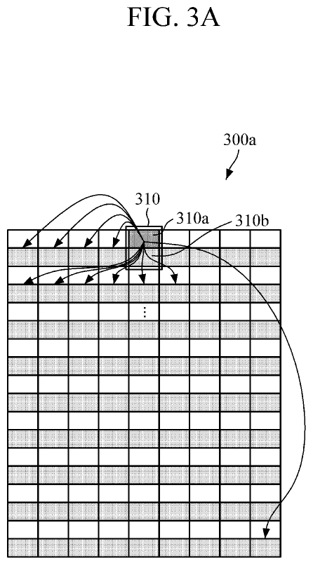

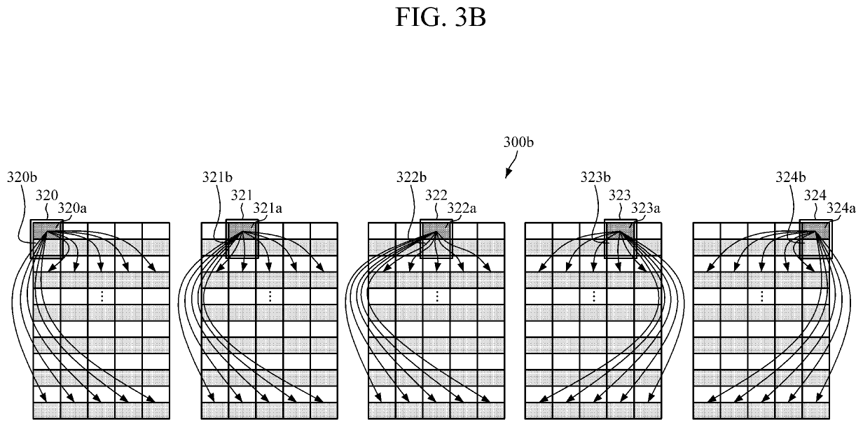

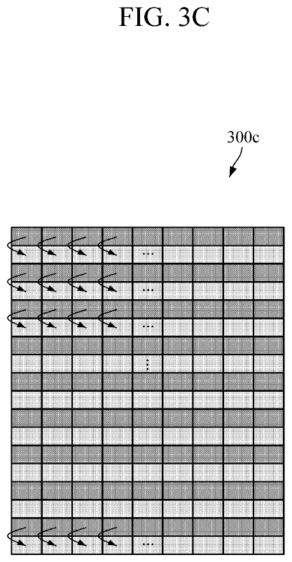

Various driving methods of the light sensor 111 will be described with reference to FIGS. 3A to 3C. FIGS. 3A to 3C are diagrams for explaining a process in which a light source and a detector are driven in a light sensor formed of a pixel array.

FIG. 3A illustrates a 9×9 pixel array of the light sensor 111. Referring to FIG. 3A, the light sensor controller 121 may drive the light sensor 111 according to a first driving method. For example, a light source 310a of a specific pixel 310 in a pixel array 300a may be driven to emit light to an object, and at this time, the detectors of all pixels including a detector 310b of the specific pixel 310 may be driven and a light signal scattered or reflected from the object may be detected by the detector of each pixel. Accordingly, light signals on different light paths may be detected.

FIG. 3A illustrates that the light source 310a of the pixel 310 is placed in the first row and the fifth column is driven, but the disclosure is not limited thereto and a light source of any pixel in the pixel array 300a may be driven.

Referring to FIG. 3B, the light sensor controller 121 may drive the light sensor 111 according to a second driving method. For example, the light sensor controller 121 may determine a plurality of light source driving pixels in which light sources are to be driven in the pixel array 300b, drive the light sources of the determined pixels, and drive detectors of pixels other than the determined light source driving pixels.

For example, referring to FIG. 3B, the light sensor controller 121 may determine pixels 320, 321, 322, 323, and 324 on a first row to be light source driving pixels. The pixels 320, 321, 322, 323, and 324 determined to be the light source driving pixels may each include light sources 320a, 321a, 323a, 322a, and 324a and detectors 320b, 321b, 322b, 323b, and 324b, respectively, as illustrated.

In this case, the light sensor controller 121 may drive the light source 320a of the pixel 320 in the first row and a first column, and drive detectors of pixels other than the pixels 320, 321, 322, 323, and 324 in the first row, which are light source driving pixels. Then, the light sensor controller 121 may drive the light source 321a of the pixel 321 in the first row and a second column, and drive detectors of pixels other than the pixels 320, 321, 322, 323, and 324 in the first row, which are light source driving pixels. In a similar way, the light sensor controller 121 may drive the remaining pixels 322, 323, and 324, which are determined to be the light source driving pixels, and detectors of pixels other than the pixels 320, 321, 322, 323, and 324 in the first row.

In FIG. 3B, the light sources 320a, 321a, 322a, 323a, and 324a of the pixels 320, 321, 322, 323, and 324 in the first row, which are light source driving pixels, are illustrated as being driven in order from left to right, but is not limited thereto, and the driving order of the light sources of the light source driving pixels may be changed without limitation.

In the case of FIG. 3B, the light sensor controller 121 is illustrated as determining the pixels in the first row to be the light source driving pixels, but is not limited thereto and the light sensor controller 121 may determine pixels in another row other than the first row in the pixel array 300b to be light source driving pixels, or determine pixels in a plurality of rows of the pixel array 300b to be the light source driving pixels. Alternatively, unlike FIG. 3B, the light sensor controller 121 may not determine pixels in a specific row of the pixel array 300b to be light source driving pixels, but may determine pixels in a specific column of the pixel array 300b to be light source driving pixels, or may determine arbitrary pixels in the pixel array 300b to be light source driving pixels, rather than pixels arranged side by side, such as pixels in the same row or pixels in the same column of the pixel array 300b.

Under the control of the light sensor controller 121, a light source of the pixel determined to be the light source driving pixel may emit light to the object, and at this time, the detectors of the pixels other than the light source driving pixel may detect light signals scattered or reflected by the object. Accordingly, light signals on different light paths may be detected.

Referring to FIG. 3C, the light sensor controller 121 may drive the light sensor 111 according to a third driving method. For example, the light sensor controller 121 may determine one or more light source driving pixels in which light sources are to be driven in a pixel array 300c, drive the light sources of the determined pixels, and drive detectors of pixels in which the light sources are driven.

For example, the light sensor controller 121 may first drive the light source of the pixel in a first row and a first column, and in the meantime drive the detector of the same pixel as that of the driven light source, that is, the detector of the pixel in the first row and the first column. Then, the light sensor controller 121 may drive the light source of the pixel in the first row and a second column, and in the meantime drive the detector of the same pixel as that of the driven light source, that is, the detector of the pixel in the first row and the second column.

At this time, among the pixels of the pixel array 300c, the light sensor controller 121 may drive the light source and the detector of each pixel in the order of the light source and detector of the pixel in the first row and the first column, the light source and detector of the pixel in the first row and the second column, and then the light source and detector of the pixel in the first row and the third column. However, the disclosure is not limited thereto, and a pixel of which the light source and detector are to be first driven may be determined without limitation.

FIG. 3C illustrates that the light sensor controller 121 determines all pixels of the pixel array 300c to be light source driving pixels and drives a detector of the same pixel as that of the light source driven, but the disclosure is not limited thereto, and the light sensor controller 121 may determine that only some pixels in the pixel array are light source driving pixels.

The light sensor controller 121 may determine a wavelength band of light emitted by the light source of each pixel. In this case, the light sensor controller 121 may control the light sources of each pixel or the plurality of light sources of one pixel to all emit light of the same wavelength or light of different wavelengths. For example, the light sources of each pixel or the plurality of light sources of one pixel may emit light of green, blue, red, infrared wavelength, etc., but is not limited thereto. The light signal detected by the detector may differ according to the wavelength band of the light emitted by each light source.

Referring back to FIG. 2, for example, the preprocessor 122 may perform preprocessing, such as filtering for removing noise, such as motion noise or the like, from sensor data obtained from the light sensor, amplification of the sensor data, or the like. For example, the preprocessor 122 may use a bandpass filter to perform bandpass filtering of 0.4 Hz to 10 Hz, thereby removing noise from the sensor data received from the training data collector 110. The bandpass filter may be a digital filter implemented in software code executable by the preprocessor 122. In another example, the bandpass filter may be an analog filter, and in this case, the sensor data obtained by the training data collector 110 may be transmitted to the preprocessor 122 after passing through the bandpass filter (not shown). In addition, the processor 122 may correct bio-signals through reconstruction of the bio-signals based on fast Fourier transform. However, the disclosure is not limited thereto, and various other preprocessing operations may be performed according to various measurement environments, such as the computing performance or measurement accuracy of the apparatus, the position of the object, the temperature and humidity of the object, the temperature of the sensor part, etc.

In another example, the preprocessor 122 may calculate a moving average of a sensor data variable over time for each user, which is obtained by the training data collector 110. A moving average may be a cumulative weighted moving average obtained by accumulating and weighting data in units of a predetermined moving average period.

In this case, the moving average period unit and/or a weight for each period unit may be preset. For example, the preprocessor 122 may set a moving average period unit to be 3 and may assign decreasing weights to data farther from the central point of the moving average period unit. For example, when time T is a central point of a moving average period unit, a weight of 1 may be assigned at time T−1, a weight of 2 may be assigned at time T, and a weight of 1 may be assigned at time T+1, but the disclosure is not limited thereto, and the moving average period unit and the method of setting a weight may be modified without limitation. By using such preprocessing through the cumulative weighted moving average, the influence that the noise generated in the sensor data acquisition process of the training data collector 110 has on the lipid concentration prediction model may be reduced.

In another example, the preprocessor 122 may obtain additional sensor data by augmenting the sensor data obtained by the training data collector 110. In this case, the preprocessor 122 may augment the data using various data augmentation techniques, and, for example, may augment the sensor data based on Gaussian blur using an image filter based on a normal distribution. However, the disclosure is not limited thereto, and various data augmentation techniques that can be used for augmenting sensor data, which is image data, may be used.

In this case, the additional sensor data may have a pattern similar to that of the sensor data obtained by the training data collector 110. In general, a large amount of data is required to sufficiently train a lipid concentration prediction model and improve its performance, but it takes a significant amount of time and cost to acquire the reference lipid concentration and sensor data multiple times from a plurality of users. In this way, by acquiring the additional sensor data through data augmentation, the cost and time required may be reduced and at the same time an enormous amount of training data may be acquired.

The preprocessor 122 may scale the sensor data variable obtained by the training data collector 110. The preprocessor 122 may scale the sensor data variable using, for example, an L-2 norm. For example, the preprocessor 122 may scale a sensor data variable associated with each acquisition time point based on a plurality of sensor data variables acquired at the same time point. In this case, an equation such as Equation 1 for the L-2 norm below may be used, but the disclosure is not limited thereto.

$${x}_{norm} = \frac{x}{‖ x {‖}_{2}} \qquad ( 1 )$$

Here, x denotes a sensor data variable before scaling, and xₙₒᵣₘ denotes a sensor data variable after scaling by the L-2 norm. In this case, the sensor data variable may be, for example, a feature value in each of a plurality of light signals obtained at each time point, such as, as described above, an average of AC component amplitudes, an average of DC component amplitudes, an average value obtained by dividing an AC component by a DC component, an area value, or a maximum value, a minimum value, or a statistical value of a maximum value and a minimum value in a differential signal.

The valid variable selector 123 may select a valid variable significant (or substantially relevant) to the change in lipid concentration based on the training data collected by the training data collector 110 or the data preprocessed by the preprocessor 122. In this case, the valid variable selector 123 may select a valid variable from among metadata variables and/or sensor data variables.

For example, the valid variable selector 123 may select a valid variable using a nonparametric statistical test including a Wilcoxon rank-sum test based on the training data.

The valid variable selector 123 may classify the collected training data into a first group having a reference lipid concentration greater than or equal to a first threshold and a second group having a reference lipid concentration less than or equal to a second threshold. In this case, the first group may be a group having a reference lipid concentration greater than or equal to the third quartile in the distribution of the obtained reference lipid concentration, and the second group may be a group having a reference lipid concentration less than or equal to the first quartile in the distribution of the obtained reference lipid concentration. However, the disclosure is not limited thereto.

In this case, the valid variable selector 123 may compare the sensor data and/or metadata of the first group and the second group, and select, as a valid variable, a variable determined to be significant to the change in lipid concentration from among the sensor data variables and the metadata variables.

In another example, the valid variable selector 123 may select a valid variable using an auto-encoder based on the training data collected by the training data collector 110.

In general, an auto-encoder may include an unsupervised learning-based artificial neural network model that is trained so that a desired output approximates to an input, and may include an encoding process and a decoding process. The valid variable selector 123 may encode a sensor data variable input to an input layer to a hidden layer using only an encoding process of the auto-encoder, thereby selecting a valid variable significant to the change in lipid concentration. That is, the valid variable selector 123 may encode the variable having high dimensional data by using an auto-encoder, thereby compressing the input variable and selecting a predetermined valid variable having smaller dimensional data.

The lipid concentration prediction model generator 124 may generate a lipid concentration prediction model based on the training data collected by the training data collector 110 and the valid variable selected. In this case, the lipid concentration prediction model generator 124 may use various types of machine learning models. The machine learning models may include linear models and nonlinear models, and the machine learning models may include, for example, at least one of partial least square (PLS), elastic net, random forest, gradient boosting machine (GBM), or XGBoost, but are not limited thereto.

The lipid concentration prediction model generator 124 may generate a lipid concentration prediction model, such as Equation 2 below, but is not limited thereto. The lipid concentration prediction model may be defined as various linear or non-linear combination functions, such as addition, subtraction, division, multiplication, logarithmic value, regression equation, and the like, with no particular limitation. Equation 2 below represents a simple linear function.

y=ax₁+bx₂+cx₃+d (2)

In Equation 2, y denotes a lipid concentration to be estimated, and x₁, x₂, and x₃ values may each denote a selected valid variable or a value obtained by combining two or more of the selected valid variables. d denotes a fixed constant, and a, b, and c may be coefficients for weighting the selected valid variables and be fixed values that are universally applicable to a plurality of users predefined.

The storage 130 may store therein various items of reference information required for generating a lipid concentration prediction model, the obtained sensor data, the preprocessing result of the sensor data, the selection result of valid variable, and the like. In this case, the reference information may include user information, such as a user's age, gender, occupation, health condition, and the like, and information regarding a relationship between the valid variable and the lipid concentration prediction model, etc., but is not limited thereto. In this case, the storage 130 may include at least one storage medium of a flash memory type memory, a hard disk type memory, a multimedia card micro type memory, a card type memory (e.g., an SD memory, an XD memory, etc.), a random access memory (RAM), a static random access memory (SRAM), a read only memory (ROM), an electrically erasable programmable read only memory (EEPROM), a programmable read only memory (PROM), a magnetic memory, a magnetic disk, and an optical disk, and the like, but is not limited thereto.

The output interface 140 may provide the user with the sensor data, metadata, and reference lipid concentration collected by the training data collector 110 and the processing result of the processor 120. The output interface 140 may provide the information to the user in various visual/non-visual manners using a display module, a speaker, and a haptic device mounted in the apparatus.

For example, the output interface 140 may visually display the generated lipid concentration prediction model along with the selected valid variable. The output interface 140 may provide the user with the moving average, data augmentation, and scaling result for the sensor data.

The communication interface 150 may be connected to an external device through communication techniques under the control of the processor 120 and may receive a bio-signal and a reference lipid concentration from the external device. In this case, the external device may include, without limitation, various devices, such as smartphones, tablet PCs, wearable devices, and the like, which measure a bio-signal and a reference lipid concentration directly from the user or manage the measured bio-signal and reference lipid concentration. Also, the communication interface 150 may transmit the processing results of the processor 120 to the external device.

In this case, the communication techniques may include Bluetooth communication, Bluetooth Low Energy (BLE) communication, near field communication unit, WLAN communication, ZigBee communication, infrared data association (IrDA) communication, Wi-Fi direct (WFD) communication, ultra wideband (UWB) communication, Ant+communication, WI-FI communication, and mobile communication techniques, but are not limited thereto.

When both the light sensor 111 and the communication interface 150 are included in the apparatus 200 for estimating lipid concentration, the processor 120 may selectively control the light sensor 111 and the communication interface 150 to obtain a bio-signal. The light sensor 111 may be omitted according to the characteristics of the apparatus 200.

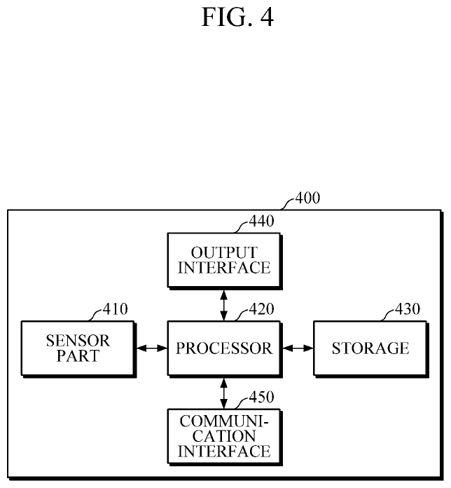

FIG. 4 is a block diagram illustrating an apparatus 400 for estimating lipid concentration according to an example embodiment.

Referring to FIG. 4, an apparatus 400 for estimating lipid concentration may include a sensor part 410, a processor 420, a storage 430, an output interface 440, and a communication interface 450.

The sensor part 410 may include a light sensor and may use the light sensor to obtain a plurality of bio-signals of a specific user for a predetermined time period. In this case, the light sensor may be formed of a pixel array, and each pixel of a pixel array may include one or more light sources and detectors, but is not limited thereto. The bio-signal obtained by the sensor part 410 may include a bio-signal for calibration and a bio-signal for lipid concentration estimation.

When a specific user estimates a lipid concentration using the apparatus for estimating lipid concentration, calibration for generating a personalized lipid concentration prediction model may be carried out. The processor 420 may perform a calibration at preset intervals, or according to an analysis of lipid concentration estimation result or a user's request.

For example, the processor 420 may perform a calibration at a time of initial use of the apparatus by the user and at preset intervals from the time of initial use. For example, when the user requests estimation of an initial lipid concentration using the apparatus 400, the processor 420 may refer to the storage 430 to check whether reference information for estimation of lipid concentration exist, and, if there is no reference information, may perform a calibration.

In another example, the processor 420 may analyze the lipid concentration estimation result and determine whether to perform a calibration based on the analysis result. For example, once the lipid concentration estimation is complete, the processor 420 may determine the accuracy of the estimated lipid concentration and determine whether to perform a calibration. For example, a normal range of an estimated lipid concentration value may be predefined, and a determination may be made based on the normal range of the estimated lipid concentration value. For example, it may be determined that a calibration is needed when an estimated lipid concentration value falls outside the normal range, when the number of times that an estimated lipid concentration value is outside the normal range is outside a threshold, when the number of consecutive occurrences in which an estimated lipid concentration value does not fall in the normal range is greater than or equal to a predetermined threshold, or when the number of occurrences in which the estimated lipid concentration value does not fall within the normal range in a predetermined time period is greater than or equal to a predetermined threshold.

When the processor 420 determines to perform a calibration, the processor 420 may control the sensor part 410 to obtain a bio-signal for calibration. For example, the processor 420 may guide the user to bring an object into contact with the sensor part 410. In this case, the processor 420 may guide the user to make the object in contact with the sensor part 410 in units of a predetermined time, for a predetermined period of time.

The processor 420 may generate a personalized lipid concentration prediction model based on the lipid concentration prediction model stored in the storage 430 or the lipid concentration prediction model received from an external source through the communication interface 450 by performing a calibration based on the bio-signal for calibration obtained by the sensor part 410 and the metadata of a specific user.

For example, the processor 420 may obtain a valid variable of the lipid concentration prediction model, for example, an average of AC component amplitudes of a first light signal, based on a bio-signal at a time of initial calibration performed in the stable state, and obtain a measured lipid concentration of the specific user at the time of calibration. In this case, the measured lipid concentration may be measured directly from the apparatus 400 for estimating lipid concentration, or received from an external device (not shown), and the stable state may refer to a state in which there is no influence of external noise and the user's physical state maintains a value within a certain error range, and may mean, for example, a fasting period. The processor 420 may apply the obtained valid variable value to the lipid concentration prediction model, perform a calibration by comparing the lipid concentration prediction model with the measured lipid concentration of the specific user, and generate a personalized lipid concentration prediction model. The generated personalized lipid concentration prediction model may be stored in the storage 430.

When a request for estimation of lipid concentration is received at the time of lipid concentration estimation, the processor 420 may estimate a lipid concentration of the user using the bio-signal for lipid concentration estimation obtained through the sensor part 410, the user's metadata, and the personalized lipid concentration prediction model.

For example, the processor 420 may extract a value corresponding to the valid variable from the bio-signal for lipid concentration estimation and/or metadata obtained through the sensor part 410, and apply the extracted valid variable value to the personalized lipid concentration model to estimate lipid concentration.

In this case, the output interface 440 may provide the estimated lipid concentration to the user in various visual/non-visual manners. Also, the output interface 440 may provide the estimated lipid concentration value to the user by using one or more of various methods, such as by changing a color, a line thickness, font, and the like based on whether the estimated lipid concentration value falls within or outside a normal range. Additionally, the output interface 440 may also use vibrations and/or tactile sensations according to an abnormal lipid concentration value being estimated so that the user can easily recognize the abnormality of the lipid concentration. Alternatively, upon comparing the estimated lipid estimation value with a previous measurement history, if it is determined that the estimated lipid concentration is abnormal, the output interface 440 may display information on a user's action to be taken, such as food information that the user should be careful about, information on a related hospital, and the like, together with a warning message, an alert signal, or the like.

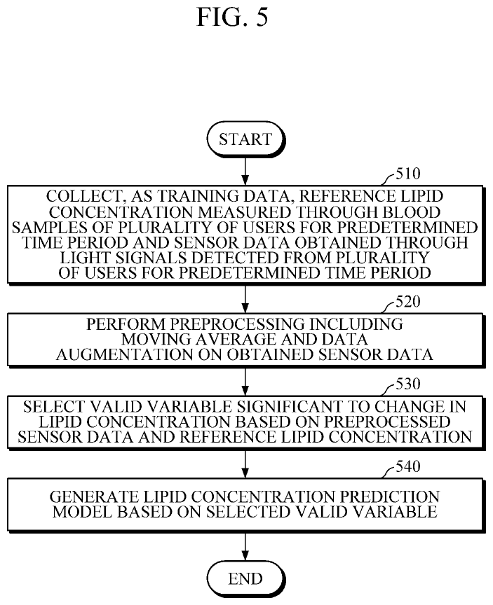

FIG. 5 is a flowchart illustrating a method of estimating lipid concentration according to an example embodiment. The method of FIG. 5 is one example embodiment of a method performed by the apparatus for estimating lipid concentration according to the example embodiment of FIG. 1 or 2, which is described above in detail such that a description thereof will be briefly given below.

First, sensor data obtained through a reference lipid concentration measured through blood samples of a plurality of users for a predetermined time period and light signals detected from the plurality of users for the predetermined time period may be collected as training data in 510.

In this case, metadata including at least one of the user's gender, age, height, weight, BMI, body fat mass, muscle mass, body water content, skin temperature, and skin humidity may be further collected as training data.

Next, preprocessing including a moving average and data augmentation may be performed on the obtained sensor data in 520.

For example, sensor data variables acquired over time for each user may be preprocessed by using the cumulative weighted moving average, wherein a lower weight may be assigned to data farther from the central point of a predetermined moving average period unit. In another example, additional sensor data may be acquired by augmenting data using a data augmentation technique including Gaussian blur. Alternatively, the sensor data variable may be scaled using the L-2 norm.

Next, a valid variable significant (or substantially relevant) to the change in lipid concentration may be selected based on the preprocessed sensor data and the reference lipid concentration in 530.

For example, the collected training data may be classified into two or more groups based on the reference lipid concentration, and a valid variable may be selected by comparing the training data between the classified groups. In this case, the valid variable may be selected by applying a nonparametric statistical test including a Wilcoxon rank-sum test to the training data in the classified groups.

In another example, the valid variable may be selected using an auto-encoder based on the training data.

Next, a lipid concentration prediction model may be generated based on the selected valid variable in 540. In this case, the lipid concentration prediction model may be generated based further on a machine learning model including at least one of PLS, elastic net, random forest, GBM, or XGBoost.

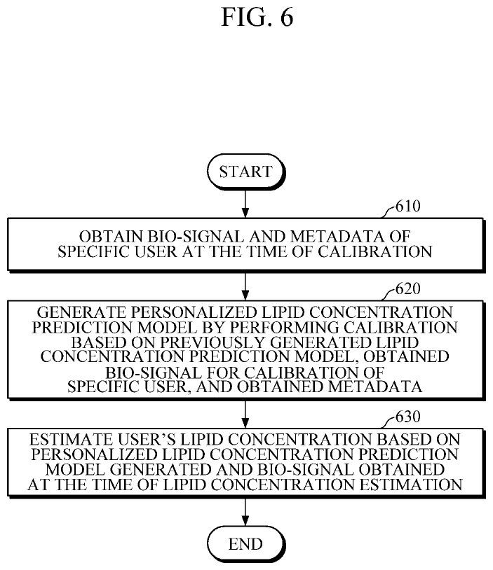

FIG. 6 is a flowchart illustrating a method of estimating lipid concentration according to an example embodiment. The method of FIG. 6 is one example embodiment of a method performed by the apparatus for estimating lipid concentration according to the example embodiment of FIG. 4, which is described above in detail such that a description thereof will be briefly given below.

First, a bio-signal and metadata of a specific user may be obtained at a time of calibration in 610. In this case, the bio-signal of the specific user may be obtained by detecting a light signal using a light sensor.

Next, in 620, a personalized lipid concentration prediction model may be generated by performing a calibration based on a previously generated lipid concentration prediction model, an obtained bio-signal for calibration of the specific user, and the obtained metadata.

Then, a user's lipid concentration may be estimated based on the generated personalized lipid concentration prediction model and the bio-signal obtained at the time of lipid concentration estimation in 630. In this case, the user's lipid concentration may be estimated based further on the user's metadata obtained at the time of lipid concentration estimation.

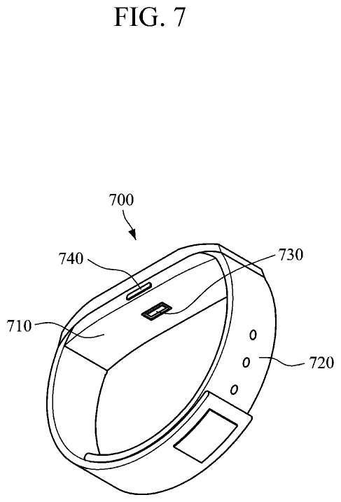

FIG. 7 is a diagram illustrating a wearable device according to an example embodiment. Various example embodiments of the apparatus 400 for estimating lipid concentration may be mounted in a smartwatch that is worn on a wrist of a user. However, the shape of the wearable device is not limited to the illustrated example.

Referring to FIG. 7, a wearable device 700 includes a main body 710 and a strap 720.

The strap 720 may be made of a flexible material. The strap 720 may be connected to opposite ends of the main body 710 and may wrap around the user's wrist such that the main body 810 is in close contact with an upper portion of the wrist. In this case, air may be injected into the strap 720 or an airbag may be included in the strap 720, so that the strap 720 may have elasticity according to a change in pressure applied to the wrist, and the change in pressure of the wrist may be transmitted to the main body 710.

A battery, which supplies power to the wearable device 700, may be embedded in the main body 710 or the strap 720. Further, a sensor part 730 may be mounted in a rear surface of the main body 710. The sensor part 730 may include a light sensor, the light sensor may be formed of a pixel array, and each pixel of the pixel array may include one or more light sources and detectors. However, the disclosure is not limited thereto.

The processor is mounted inside the main body 710 and may generate a personalized lipid concentration prediction model based on a bio-signal for calibration obtained by the sensor part 730, or estimate the user's lipid concentration based on a bio-signal for lipid concentration estimation.

In addition, a display may be mounted on the front surface of the main body 710 and may display a lipid concentration estimation result and the like. In this case, the display may include a touch screen which allows touch input.

In addition, a storage may be mounted inside the main body 710, and a lipid concentration prediction model generated in advance, a personalized lipid concentration prediction model generated as a result of calibration, and/or a processing result of the processor may be stored in the storage.

In addition, a manipulator 740 configured to receive a control command from a user and transmit the control command to the processor may be mounted on the side of the main body 710. The manipulator 740 may have a function for inputting a command to turn on/off the wearable device 700. Also, the manipulator 740 may include a PPG sensor to obtain a bio-signal from a finger when the finger is in contact with the manipulator 740.

Further, a communication interface configured to transmit and receive data with an external device may be mounted in the main body 710. The communication interface may communicate with the external device, such as the user's smartphone, a lipid concentration measuring device, or the like, to transmit and receive various types of data related to estimation of lipid concentration. The communication interface may transmit a personalized lipid concentration prediction model of a specific user generated as a result of calibration to an external database, and may periodically receive a modified lipid concentration prediction model from the external database. The processor may update the personalized lipid concentration prediction model of a specific user by performing a re-calibration based on the modified lipid concentration prediction model.

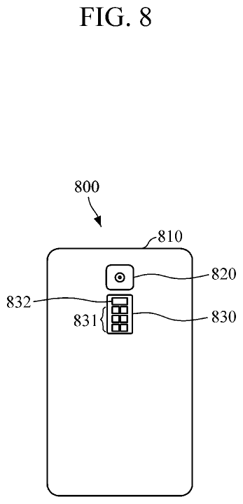

FIG. 8 is a diagram illustrating a smart device according to an example embodiment. Here, a smart device 800 may include a smartphone, a tablet PC, etc. The smart device 800 may include the above-described various example embodiments of the apparatus 400 for estimating lipid concentration.

Referring to FIG. 8, the smart device 800 may have a sensor part 830 mounted on a rear surface of a main body 810. The sensor part 830 may include a light source 831 and a detector 832. The number and arrangement of the light sources 831 and detectors 832 included in the sensor part 830 may be varied without limitation to the example shown in FIG. 8. The sensor part 830 may be mounted on the rear surface of the main body 810 as illustrated, but is not limited thereto. For example, the sensor part 830 may be formed on a fingerprint sensor on a front surface, a part of a touch panel, or a power button or volume button mounted on the side or an upper portion of the smart device.

In addition, a display for displaying various types of information, such as a lipid concentration estimation result, contact state guide information, and the like, may be mounted on the front surface of the main body 810.

An image sensor 820 may be mounted in the main body 810 as illustrated, and the image sensor 820 may capture an image of a finger when the user approaches the sensor part 830 to measure a bio-signal and may transmit the image to the processor. In this case, the processor may identify a relative position of the finger relative to the actual position of the sensor part 830 and perform operation to guide the user for information on the relative position of the finger through the display.

The processor may generate a personalized lipid concentration prediction model by performing a calibration based on a previously generated lipid concentration prediction model, a specific user's bio-signal obtained at the time of calibration, and metadata, as described above, or may estimate the user's lipid concentration based on the generated personalized concentration prediction model, the bio-signal obtained at the time of lipid concentration estimation, and the metadata. A detailed description thereof will be omitted.

The current embodiments may be implemented as computer readable codes in a computer readable record medium. Codes and code segments constituting the computer program may be easily inferred by a skilled computer programmer in the art. The computer readable record medium includes all types of record media in which computer readable data are stored. Examples of the computer readable record medium include a ROM, a RAM, a CD-ROM, a magnetic tape, a floppy disk, and an optical data storage. Further, the record medium may be implemented in the form of a carrier wave such as Internet transmission. In addition, the computer readable record medium may be distributed to computer systems over a network, in which computer readable codes may be stored and executed in a distributed manner.

At least one of the components, elements, modules or units (collectively “components” in this paragraph) represented by a block in the drawings may be embodied as various numbers of hardware, software and/or firmware structures that execute respective functions described above, according to an example embodiment. According to example embodiments, at least one of these components may use a direct circuit structure, such as a memory, a processor, a logic circuit, a look-up table, etc. that may execute the respective functions through controls of one or more microprocessors or other control apparatuses. Also, at least one of these components may be specifically embodied by a module, a program, or a part of code, which contains one or more executable instructions for performing specified logic functions, and executed by one or more microprocessors or other control apparatuses. Further, at least one of these components may include or may be implemented by a processor such as a central processing unit (CPU) that performs the respective functions, a microprocessor, or the like. Two or more of these components may be combined into one single component which performs all operations or functions of the combined two or more components. Also, at least part of functions of at least one of these components may be performed by another of these components. Functional aspects of the above exemplary embodiments may be implemented in algorithms that execute on one or more processors. Furthermore, the components represented by a block or processing steps may employ any number of related art techniques for electronics configuration, signal processing and/or control, data processing and the like.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An apparatus for estimating lipid concentration, comprising:
a training data collector configured to collect, as training data, a reference lipid concentration measured through blood samples of a plurality of users for a predetermined time period and sensor data obtained through light signals detected from the plurality of users for the predetermined time period; and
a processor configured to perform preprocessing, including a moving average and data augmentation, on the obtained sensor data, configured to select a valid variable relevant to a change in lipid concentration based on the preprocessed sensor data and the reference lipid concentration, and configured to generate a lipid concentration prediction model based on the selected valid variable.

2. The apparatus of claim 1, wherein the training data collector is further configured to collect, as the training data, metadata including at least one of gender, age, height, weight, body mass index (BMI), skin temperature, or skin humidity of the plurality of users, and the processor is further configured to select the valid variable based further on the collected metadata.

3. The apparatus of claim 1, wherein the processor is further configured to perform the preprocessing on a sensor data variable obtained over time for each user of the plurality of users using a cumulative weighted moving average, wherein a lower weight is assigned to data farther from a central point of a predetermined moving average period unit.

4. The apparatus of claim 1, wherein the processor is further configured to obtain additional sensor data by augmenting data based on the sensor data using a data augmentation technique including Gaussian blur.

5. The apparatus of claim 1, wherein the processor is further configured to scale a senor data variable using an L-2 norm.

6. The apparatus of claim 1, wherein the processor is configured to classify the collected training data into at least two groups based on the reference lipid concentration and select the valid variable by comparing the training data between the classified at least two groups.

7. The apparatus of claim 6, wherein the processor is further configured to select the valid variable by applying a nonparametric statistical test including a Wilcoxon rank-sum test to the training data in the classified at least two groups.

8. The apparatus of claim 1, wherein the processor is further configured to select the valid variable using an auto-encoder based on the training data.

9. The apparatus of claim 1, wherein the processor is further configured to generate the lipid concentration prediction model based further on a machine learning model including at least one of partial least square (PLS), elastic net, random forest, gradient boosting machine (GBM), or XGBoost.

10. The apparatus of claim 1, wherein the training data collector comprises a light sensor provided in a pixel array, the pixel array comprising light sources configured to emit light toward an object and detectors configured to detect a light signal through light scattered or reflected from the object.

11. The apparatus of claim 10, wherein the processor is further configured to drive a light source of a specific pixel and detectors of all pixels in the light sensor.

12. The apparatus of claim 10, wherein the processor is further configured to sequentially drive light sources of pixels in a specific row of the pixel array and drive detectors in remaining rows of the pixel array while the light sources of the pixels in the specific row are being sequentially driven.

13. The apparatus of claim 10, wherein the processor is further configured to sequentially drive light sources of all pixels of the pixel array and drive a detector of the same pixel as that of a driven light source while the light sources of all pixels are being sequentially driven.

14. The apparatus of claim 1, wherein the processor is further configured to generate a personalized lipid concentration prediction model by performing a calibration based on the generated lipid concentration prediction model, a bio-signal obtained through a light signal detected from a specific user, and metadata of the specific user.

15. A method of estimating lipid concentration, comprising:
collecting, as training data, a reference lipid concentration measured through blood samples of a plurality of users for a predetermined time period and sensor data obtained through light signals detected from the plurality of users for the predetermined time period;
performing preprocessing including a moving average and data augmentation on the obtained sensor data;
selecting a valid variable relevant to a change in lipid concentration based on the preprocessed sensor data and the reference lipid concentration; and
generating a lipid concentration prediction model based on the selected valid variable.

16. The method of claim 15, wherein the collecting comprises further collecting, as the training data, metadata including at least one of gender, age, height, weight, body mass index (BMI), skin temperature, or skin humidity of the plurality of users and the selecting of the valid variable comprises selecting the valid variable based further on the collected metadata.

17. The method of claim 15, wherein the performing the preprocessing comprises obtaining additional sensor data by augmenting data based on the sensor data using a data augmentation technique including Gaussian blur.

18. The method of claim 15, wherein the selecting the valid variable comprises classifying the collected training data into at least two groups based on the reference lipid concentration and selecting the valid variable by comparing the training data between the classified at least two groups.

19. The method of claim 18, wherein the selecting the valid variable by comparing the training data between the classified at least two groups comprises selecting the valid variable by applying a nonparametric statistical test including a Wilcoxon rank-sum test to the training data in the classified at least two groups.

20. The method of claim 15, wherein the selecting of the valid variable comprises selecting the valid variable using an auto-encoder based on the training data.
